(19) United States
(12) Patent Application Publication
(10) Pub. No.: US 2024/0024401 A1
(43) Pub. Date: Jan. 25, 2024

(54) POLYSACCHARIDE COMPOSITION AND METHOD FOR HEALTH MAINTENANCE AND STRENGTHENING THE IMMUNE SYSTEM

(71) Applicant: Kodimule Shyam PRASAD, Bangalore (IN)

(72) Inventor: Kodimule Shyam PRASAD, Bangalore (IN)

(21) Appl. No.: 18/224,218

(22) Filed: Jul. 20, 2023

Related U.S. Application Data
(60) Provisional application No. 63/391,799, filed on Jul. 25, 2022.

(51) Int. Cl. A61K 36/51; A61P 31/00; A23L 33/105

(52) U.S. Cl. CPC A61K 36/51; A61P 31/00; A23L 33/105

(57) ABSTRACT

The invention provides a polysaccharide-rich composition comprising coffee fruit extract. The composition produces an immunostimulatory effect and can increase the activity of macrophages. The composition finds use in methods of stimulating an immune response and supporting immune system function in a subject.

### CROSS-REFERENCE TO RELATED APPLICATIONS

This application claims the benefit of provisional application No. 63/391,799 filed Jul. 25, 2022, the entire contents of which are incorporated herein by reference for all purposes.

### FIELD OF THE INVENTION

The invention generally relates to herbal compositions. More particularly, the invention relates to a composition comprising coffee pulp extract and method of its use in health maintenance and strengthening the immune system.

### BACKGROUND

The immune system regulates various physiological processes that help a host to defend itself against infections of bacteria, viruses, fungi and parasites. Therefore, improving the body's immunity and strengthening the immune function is crucial to preventing disease onset and maintaining and restoring health. Since immunity is fundamental to host health, systematic studies are necessary to explore natural biomolecules that can enhance innate and adaptive immune systems by activating immune cells and improving immunomodulatory molecules.

Polysaccharides are natural biomacromolecules consisting of various monosaccharides linked through glycosidic linkages with uronic acids. Polysaccharides are drawing attention for health maintenance and treatments owing to their low toxicity, lack of adverse effects, and their various biological activities, including antioxidant, antiviral, anti-fatigue, anticancer, hypoglycemic, and anti-inflammatory effects (Balan, Rozewski, Zdanowski, & Skopinska-Rozewska, 2012).

What is needed in the art therefore is a polysaccharide composition and method of its use for health maintenance and strengthening the immune system without triggering adverse side effects.

### SUMMARY OF THE INVENTION

Coffee (Coffea arabica L.) belongs to the Rubiaceae family and is a popular beverage worldwide. The inventor surprisingly discovered that the coffee plant can provide a rich source of polysaccharides capable of producing an immunostimulatory effect and concomitant health maintenance.

Thus, it is an object of the invention to provide a composition comprising an extract of coffee fruit pulp having at least 50% w/w of one or more polysaccharides.

In some aspects, the composition further comprises one or more excipients.

In some aspects, the one or more excipients include artificial excipients.

In aspects of the invention, the composition is combined with a food, beverage or nutritional supplement.

In other aspects of the invention, the composition is in a form selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, drop, topical patch, buccal patch, bead, gummy, gel, sol, injection, or combinations thereof.

It is a further object of the invention to provide a method of supporting the immune system, comprising administering to a subject in need thereof an effective amount of a composition as disclosed herein.

It is a further object of the invention to provide a method of strengthening the immune system, comprising administering to a subject in need thereof an effective amount of a composition as disclosed herein.

In another aspect, the invention provides a method of boosting the immune system, comprising administering to a subject in need thereof an effective amount of a composition as disclosed herein.

In yet another aspect, the invention provides a method of stimulating the immune system, comprising administering to a subject in need thereof an effective amount of a composition as disclosed herein.

In still other aspects, the invention provides a method of promoting overall health, comprising administering to a subject in need thereof an effective amount of a composition as disclosed herein.

Under the forgoing methods, the subject can be infected with one or more pathogens, wherein administering the composition lessens the symptoms of the pathogen infection, inhibits or prevents the progression of the symptoms of the pathogen infection, or shortens the duration of the pathogen infection.

In some aspects of the forgoing methods of the invention, administering the composition prevents the subject from becoming infected with a pathogen.

The pathogen can be one or more bacteria, one or more viruses, one or more fungi, one or more parasites, or combinations thereof.

The composition can be administered systemically or topically.

The composition can be administered orally.

The composition can be administered to the subject in one or more dosage forms selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, drop, topical patch, buccal patch, bead, gummy, gel, sol, and injection.

The composition can be administered at a dose of about 100 mg/kg body weight, about 200 mg/kg body weight, about 250 mg/kg body weight, or about 300 mg/kg body weight.

### BRIEF DESCRIPTION OF THE DRAWINGS

FIG. 1 shows the effect of an embodiment of the inventive composition on the viability of RAW 264.7 macrophages. The cells were treated with various concentrations of the inventive composition for 24 hours. Cell viability was assessed with an MTT reduction assay, and the results were expressed as the mean±SE of three independent experiments.

FIG. 2 shows the effect of an embodiment of the inventive composition on phagocytosis of normal RAW264.7 cells. Data are presented as mean±SE. Different lowercase alphabet letters were significantly different if p<0.05.

FIG. 3 shows the effect of an embodiment of the inventive composition on the production of NO in RAW 264.7 macrophages. The cells were treated with indicated concentrations of the inventive composition for 24 hours. The levels of NO in culture media were measured by Griess assay and the results were expressed as the mean±SE of three independent experiments. *P<0.05 compared to the control.

FIG. 4 shows the effect of an embodiment of the inventive composition on release of cytokines (A) TNF-α; (B) IL-6, (C) IL-1β and (D) Il-10, in RAW 264.7 cells with LPS (1 μg/ml) as a positive control. Data represent Mean±SEM of triplicate.

FIG. 5 shows the effect of an embodiment of the inventive composition on iNOS and COX2 in RAW 264.7 macrophages. The cells were treated with the indicated concentrations of the inventive composition and positive control LPS 1 μg/mL for 24 hours. Cell lysates were prepared for western blot analysis with antibodies specific for murine iNOS and COX-2.

### DEFINITIONS

As used herein, the term “subject” includes warm-blooded animals, preferably mammals, including humans.

As used herein, the phrase “effective amount” includes an amount effective, at dosages and for periods of time necessary, to achieve the desired result in a subject. An effective amount of a composition of the invention, as disclosed herein, may vary according to factors such as the disease state, age, and weight of the subject, and the ability of the composition to elicit a desired response in the subject. Dosage regimens may be adjusted to provide the optimum therapeutic response. An effective amount is also one in which any toxic or detrimental effects (e.g., side effects) of the compound are outweighed by the beneficial effects.

As used herein, the phrase “in need thereof” refers to a subject that requires the therapeutic or health benefit effects for which the composition is administered as a result of a disease, disorder or deficiency in the subject. “In need thereof” can, but does not necessarily, refer to a subject that has been diagnosed with, or determined to be as risk of having or developing, a disease, condition or disorder for which the composition is being administered. “In need thereof” can also refer to a subject that merely desires the potential benefits or effects for which the composition is administered.

As used herein, the term “about” means the numerical quantity, value or amount that is referenced, or that varies (plus or minus) by up to 5%, up to 10%, up to 15%, or up to 20% of the referenced numerical quantity, value or amount.

As used herein, the terms “inhibit,” “inhibits,” “inhibiting,” “inhibited,” and the like mean slowing, but not completely halting, the progression of, or increase in, the referenced condition or parameter as a result of the effects of the inventive composition relative to control conditions that lack the involvement of the inventive composition.

As used herein, the terms “arrest,” “arrests,” “arresting,” “arrested,” and the like mean halting or maintaining the referenced condition or parameter at a static level as a result of the effects of the inventive composition relative to control conditions that lack the involvement of the inventive composition.

As used herein, the terms “delay,” “delays,” “delaying,” “delayed,” and the like mean forestalling the appearance of the referenced condition or parameter for a period of time as a result of the effects of the inventive composition relative to control conditions that lack the involvement of the inventive composition.

As used herein, the terms “prevent,” “prevents,” “preventing,” “prevented,” and the like mean keeping the referenced condition or parameter from appearing or expressing itself as a result of the effects of the inventive composition relative to control conditions that lack the involvement of the inventive composition.

### DETAILED DESCRIPTION

The inventor surprisingly discovered a polysaccharaide composition having efficacy in modulating the immune system. The composition finds use in methods of supporting the immune system, strengthening the immune system, boosting the immune system, stimulating the immune system, and providing and supporting overall health.

In a non-limiting embodiment, the composition comprises an extract of coffee fruit having one or more polysaccharides. The extract can comprise at least about 50% w/w of the one or more polysaccharides. The extract can comprise at least 50% w/w of the one or more polysaccharides. In some embodiments, the extract comprises 50% w/w of the one or more polysaccharides.

Coffee fruit extracts for use with the invention can be obtained from the coffee fruit of one or more species of coffee. Suitable, non-limiting examples of coffee species for use with the invention include Coffea arabica (e.g., Coffea arabica L.), Coffea canephora, Coffea stenophylla, Coffea canephora, Coffea excelsa, Coffea eugenioides, Coffea sessiliflora, Coffea liberica, Coffea racemose and Coffea commersoniana. In a preferred embodiment, the composition comprises an extract of Coffea arabica L.

Coffee fruit extracts for use with the invention can be obtained from any extraction process capable of obtaining bioactive polysaccharides from coffee fruit. Suitable solvents for obtaining coffee fruit extract for use with the invention include, but are not necessarily limited to, aqueous solvents, alcohol-based solvents, supercritical fluids, polar organic solvents (such as acetone and methylethyl ketone), or combinations thereof. Non-limiting examples of alcohol-based solvents include, but are not limited to, ethanol, isopropyl alcohol, methanol, and combinations thereof. The supercritical fluid can be, but is not necessarily limited to, carbon dioxide. In some aspects of the invention, the coffee fruit extract is obtained from coffee fruit pulp. The coffee fruit pulp can be a by-product of the processing of coffee fruit for the production of green coffee beans for roasting. Coffee fruit pulp for use with the invention can be fresh, dried, or a combination thereof.

In some aspects, the composition of the invention comprises one or more excipients. Excipients for use with the inventive composition can be selected on the basis of their compatibility with one or more bioactive polysaccharides contained in the coffee fruit extract, and/or the properties of the desired dosage form. Suitable excipients include, but are not limited to, carriers, binders, fillers, flow aids/glidents, disintegrants, lubricants, stabilizers, surfactants, preservatives, diluents, and the like. The one or more excipients can be artificial excipients. Suitable excipients include, but are not necessarily limited to, those disclosed in the following references, the entire disclosures of which are incorporated herein by reference for all purposes: Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999). The composition can further comprise one or more of a sweetener, flavor, vitamin, mineral, sugar, protein, amino acid, and starch. The composition can be combined with beverages, foods, nutritional supplements, and snacks. The beverages, foods, nutritional supplements, and snacks can be dietetic.

The composition can assume any dosage form suitable for administration to a subject. Suitable dosage forms for the composition include, but are not necessarily limited to, powders, liquids, pills, tablets, pellets, capsules, thin films, solutions, sprays, syrups, linctuses, lozenges, pastilles, chewing gums, pastes, vapors, suspensions, emulsions, ointments, creams, lotions, liniments, drops, topical patches, buccal patches, beads, gummies, gels, sols and injections.

In some aspects, the composition is enclosed in a container, wherein the container includes instructions for a method of using the composition. The instructions can be printed matter. The instructions can provide information on how to use the composition for one or more of the methods disclosed herein.

In some embodiments, the invention provides a method of using the inventive composition for the purpose of modulating the immune system. The composition can be administered to a subject for the purpose of supporting the immune system, strengthening the immune system, boosting the immune system, stimulating the immune system or providing and supporting overall health. Such methods can be practiced by administering to a subject an effective amount of the inventive composition.

In some embodiments, the inventive composition is administered to a subject that is infected with one or more pathogens. The subject can be infected with one or more bacteria, one or more viruses, one or more fungi, one or more parasites, or combinations thereof. In some aspects of the invention, the subject is infected with one or more pathogens and administering the lessens the symptoms of the pathogenic infection, inhibits the progression of the symptoms of the pathogenic infection, arrests the progression of the symptoms of the pathogenic infection, or shortens the duration of the pathogenic infection. In some aspects, administering the composition to the subject prevents or delays the infection of the subject by one or more pathogens.

Without wishing to be bound by any particular theory or mechanism of action, administering the inventive composition to a subject produces an immunostimulatory effect by increasing macrophage phagocytosis through increased nitric oxide (NO) production. The inventive composition can also produce an immunostimulatory effect by stimulating macrophage secretion of proinflammatory cytokines, including TNF-α and interleukins (e.g., IL-6, IL-1β and IL-10). Thus, the inventive composition can be administered to a subject to stimulate an inflammatory response.

The methods disclosed herein can be practiced by administering an effective amount of the inventive composition by any route capable of delivering the biologically active components of the composition to the subject in a manner that permits the components to impart the therapeutic and/or health benefits disclosed herein. The composition can be administered systemically and/or locally. Suitable administration routes for the composition include, but are not limited to, auricular, buccal, conjunctival, cutaneous, dental, endocervical, endosinusal, endotracheal, enteral, epidural, extra-amniotic, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal dental, intracoronary, intracorporus cavernosum, intradermal, intradiscal, intraductal, intraduodenal, intradural, intraepidermal, intraesophageal, intragastric, intravaginal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parentera, percutaneous, periarticular, peridural, perineural, periodontal, rectal, inhalation, retrobulbar, soft tissue, subarachnoid, subconjunctival, subcutaneous, sublingual, submucosal, topical, transdermal, transmucosal, transplacental, transtracheal, transtympanic, ureteral, urethral, vaginal, or combinations thereof. In some preferred embodiments, the composition is administered orally. The composition can be administered by irrigation, drip, infusion, or topically by a dressing, patch, or bandage that is in contact with the composition.

The methods of the invention can be practiced by administering to a subject in need thereof an effective amount of the inventive composition. An effective amount of the composition (i.e., an effective dosage) may range from about 5 to about 200 mg/kg body weight, about 50 to about 200 mg/kg body weight, about 100 to about 200 mg/kg body weight, about 150 to about 200 mg/kg body weight, about 5 to about 300 mg/kg body weight, about 50 to about 300 mg/kg body weight, about 100 to about 300 mg/kg body weight, about 150 to about 300 mg/kg body weight, or about 200 to about 300 mg/kg body weight. The effective amount of the inventive composition can be about 100 mg/kg body weight, about 150 mg/kg body weight, about 200 mg/kg body weight, about 250 mg/kg body weight, or about 3000 mg/kg body weight. In some preferred embodiments, an effective amount of the composition is about 100 to about 200 mg/kg body weight. The skilled artisan will appreciate that certain factors may influence the dosage of the effective amount, including but not limited to the desired outcome, the severity of the disease or disorder, previous treatments and administrations, the general health and/or age of the subject, and other diseases present. Moreover, treatment of a subject with an effective amount of the inventive composition can include a single treatment or administration, or can include a series of treatments or administrations. It will also be appreciated that the effective amount of the composition used for the methods herein may increase or decrease over the course of a particular administration regime.

### Examples

### Materials and Methods

Dulbecco's modified eagle medium (DMEM), fetal bovine serum (FBS) and penicillin-streptomycin were purchased from Gibco-BRL® (Grand Island, NY, USA). 3-(4,5-dimethylthiazol-2-yl)-2,5-diphenyl tetrazolium bromide (MTT), LPS (Escherichia coli 0111: B4), sodium nitrite (NaNO2), Griess Reagent were purchased from Sigma-Aldrich. Antibodies were purchased from Santa Cruz.

### Sample Preparation

A standardized polysaccharide extract (the inventive composition) was obtained.

### Cytotoxicity Assay

Cytotoxicity evaluation of the inventive composition was determined by the MTT assay as described previously by Abdullah, Abdulghani, Ismail, and Abidin (2017). About 1×10⁴ cells/well (RAW 264.7) were seeded in a flat bottomed 96 well plate and were incubated for 24 hr at 37° C. in 5% CO₂ incubator. Various concentration (50-400 μg/mL) of the standardized inventive composition in medium were separately added to the plate wells. After 24 h, the supernatants were removed and subsequently, 100 μl of the MTT solution (5 mg/mL in PBS) were added to each well, to dissolve formazan crystals. The reduction of MTT was quantitated by measurement of the absorbance at 490 nm on the microplate Reader.

### Determination of Phagocytosis

Effects of the inventive composition on the phagocytosis of RAW264.7 cells were determined by using the neutral red uptake method as described by previous literature. Briefly, RAW264.7 cells (10⁵ cells/mL) were suspended in DMEM and placed in a 96-well plate. All cells were mixed with different dosages of the inventive composition (100 and 200 μg/mL). After culturing for 24 h, the supernatant was replaced with 100 μL of 0.1% neutral red solution. After incubation for 4 h, cells were washed by using 0.1 M PBS for three times to remove excess neutral red solution. At last, 100 μL of cell lysis solution (ethanol/acetic acid, 1:1, v/v) was added to each well. The absorbance of each well was measured at 540 nm by a microplate reader (Multi scan EX, Thermo Scientific).

### Measurement of Nitric Oxide Production in RAW 264.7 Macrophages

Cells (1×10⁵ cells/well seeded on a 96-well plate) were treated with the inventive composition (100-200 μg/mL) and LPS (1 μg/mL) for 24 hr and then the supernatants were reacted with same volume of Griess reagent and incubated for 15 min at room temperature. The absorbance was determined at 540 nm using microplate reader (Thermoscan EX). The calculated nitric oxide concentration was based on a nitrite calibration curve (1-100 μM).

### Determination of Levels of TNF-α, IL-6, IL-1β and IL-10

RAW264.7 cells (10⁵ cells/mL) were seeded in a 96-well plate and mixed with different dosages of the inventive composition (100 and 400 μg/mL). After incubating for 24 h, cytokines (Prostaglandin E2, TNF-α, IL-1β and IL-6) in the supernatant were measured by ELISA assay according to manufactures' instructions. The absorbance was determined at 540 nm using a microplate reader.

### Protein Extraction and Western Blot Analysis

Cells were seeded in 6-well plates at 2×10⁶ cells/well and allowed to adhere to plate for 24 hours. And then they were treated. For preparation of whole cell lysates, media was removed, and cells were washed with phosphate-buffered saline (PBS) and lysed in RIPA buffer. Proteins were separated by SDS-PAGE and transferred to nitrocellulose membranes by electroblotting. The membranes were blocked in 5% skim milk in PBS. The blots were incubated overnight at 4° C. with primary antibodies diluted in PBS followed by incubation with secondary antibodies conjugated to horseradish peroxidase for 2 h. The target protein was detected by enhanced chemiluminescence. LPS (1 μg/mL) treated group was used as a positive control.

### Statistical Analysis

Experimental values were expressed as the mean±standard error of three experiments in triplicate. Data were analysed using one-way analysis of variance, followed by post hoc Dunnett's tests. Levels of p<0.001 were considered as indicative of significance. All calculations were carried out using the GraphPad Prism.

### Results

### Effect of the Inventive Composition on Cell Viability

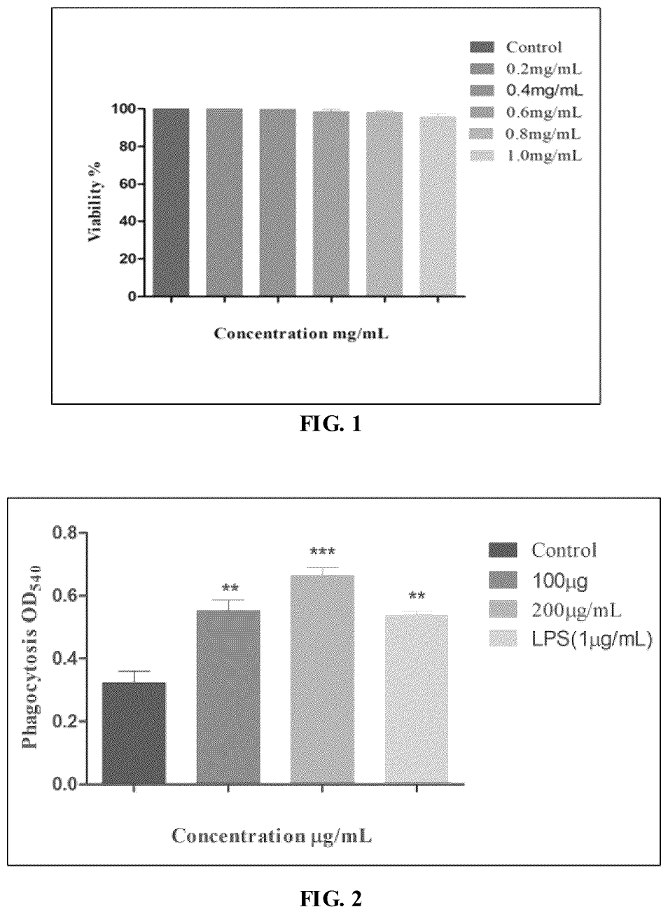

To determine the optimal concentration of the inventive composition, the RAW 264.7 cells were treated with various concentrations (0.2-1.0 mg/ml) of the inventive composition for 24 hours. The results as shown in FIG. 1 showed that different concentrations of the inventive composition did not show any toxicity effect on RAW 264.7 cells up to 1 mg/mL. There was no significant difference (p>0.05) in cell viability between the the inventive composition-treated groups and the control, which indicated that the inventive composition—had no apparent cytotoxic effect on RAW 264.7 macrophages.

### Effects of the Inventive Composition on the Phagocytosis of RAW264.7 Cells

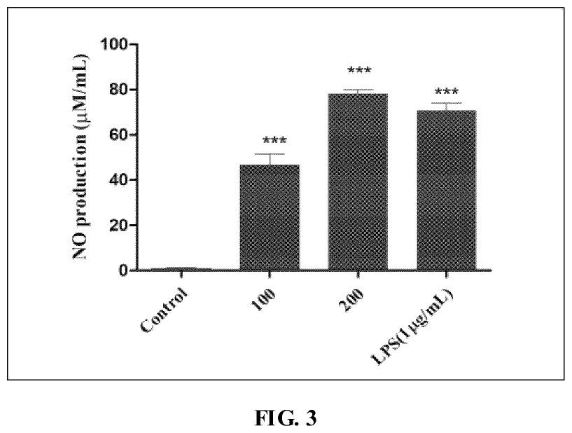

It is well known that macrophages are the most important phagocytes. The main functions of macrophages are protection against invaded pathogens through phagocytosis, presenting antigens to lymphocytes and secreting many immune mediators. Thus, phagocytosis is a crucial parameter of macrophages. Effects of the inventive composition on the phagocytosis of normal RAW264.7 cells were presented in FIG. 2. As shown in the FIG. 2, when compared to normal control group, the inventive composition treatment at the concentration of 100 μg/mL could clearly enhance the level of phagocytosis.

### NO Production of Macrophages

It has been reported that NO is a defense molecule produced by macrophages during its activation. Treatment with the inventive composition on the RAW 264.7 macrophages increased the nitrites production in a concentration-dependent manner. The results shown in FIG. 3 clearly indicated that NO production significantly increased (p>0.05) after the inventive composition treatment. At the concentration 200 μg/mL, NO level was elevated compared to unstimulated control cells and LPS treated cells.

### Increased Secretion of Cytokines in RAW 264.7 Macrophages

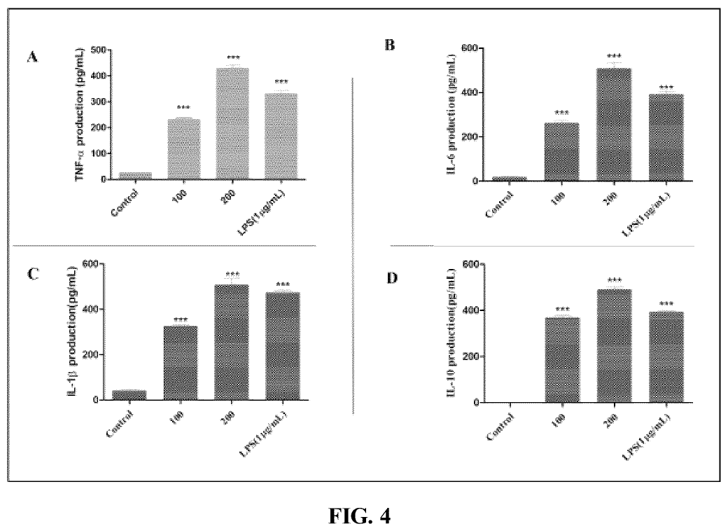

Since TNF-α and ILs (IL-6, IL-1β and IL-10) were the major proinflammatory cytokines produced by macrophages. Hence, the influence of the inventive composition on the production of cytokines of RAW 264.7 cells were studied, the levels of cytokines in the culture media were measured by ELISA. As presented in FIG. 4, the secretion of TNF-α and ILs (IL-1, IL-6, and IL-10) were significantly increased following the inventive composition treatment. At the concentration of 100 μg/mL, TNF-α and ILs (IL-1, IL-6, and IL-10) in the media of the inventive composition treated RAW 264.7 cells were higher than LPS-treated cells. These data indicate that the cytokine secreting effect of the inventive composition is found to be more than LPS. It might be due to the presence of rich polyscaccharide and other bioactive compounds present in the extract.

Effect of the Inventive Composition Inducible Nitric Oxide Synthase (iNOS) and COX-2 Expressions at the Protein Levels in RAW 264.7 Macrophages

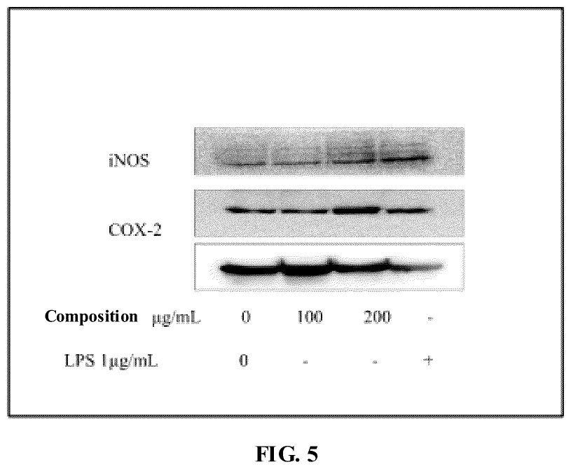

It was subsequently investigated whether the inducible effects of the inventive composition on NO production associated with protein expression of their synthetic enzymes, iNOS and COX-2, by applying western blot analysis. As shown in FIG. 5 the inventive composition effectively increased the protein expression of iNOS and COX-2, as did LPS, suggesting that the inventive composition increased NO production by inducing the expression of their encoding genes. The excessive production of NO, which are synthesized by their inducible enzymes, iNOS and COX-2, can kill normal cells and promote inflammation. However, at certain levels, they can be used as potential therapeutic tools and quantitative indexes of macrophage activation.

This is the first study about the evaluation of the immune enhancing effect of a polysaccharide-rich composition obtained from coffee fruit pulp. According to the results, the inventive composition increased the production of immune modulators, such as NO a by inducing their corresponding gene and protein expression in RAW 264.7 cells. the inventive composition also moderated the immune response by the increased release and expression of immune-related cytokines. Furthermore, enhanced the phagocytic activity of RAW 264.7 macrophages indicated that the inventive composition possessed potent immunostimulatory activity and might be treated as an attractive functional candidate for immunotherapy.

1. A method of supporting the immune system, comprising administering to a subject in need thereof an effective amount of a composition comprising at least one polysaccharide.

2. The method of claim 1, wherein the composition comprises at least 50% w/w of one or more polysaccharides.

3. The method of claim 1, wherein said composition comprises an extract of coffee fruit having at least one polysaccharide.

4. The method of claim 3, wherein said extract comprises an extract of Coffea arabica L. fruit.

5. The method of claim 3, wherein said extract is an extract of coffee fruit pulp.

6. The method of claim 3, wherein said extract comprises at least 50% w/w of said at least one polysaccharide.

7. The method of claim 6, wherein said extract includes a plurality of polysaccharides.

8. The method of claim 1, wherein said subject is infected with at least one pathogen.

9. The method of claim 8, wherein said at least one pathogen is selected from bacteria, virus, fungi, parasite, and combinations thereof.

10. The method of claim 8, wherein said composition is administered to lessen the symptoms of the pathogenic infection, inhibit the progression of the symptoms of the pathogenic infection, arrest the progression of the symptoms of the pathogenic infection, or shorten the duration of the pathogenic infection.

11. The method of claim 1, wherein said composition is administered to prevent or delay a pathogenic infection.

12. The method of claim 11, wherein said pathogenic infection is selected from bacterial infection, viral infection, fungal infection, and parasite infection.

13. The method of claim 1, wherein said composition is administered systemically or topically.

14. The method of claim 1, wherein said composition is administered by orally.

15. The method of claim 1, wherein said composition is administered in a form selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, drop, topical patch, buccal patch, bead, gummy, gel, sol, injection, or combinations thereof.

16. The method of claim 1, wherein said composition is administered at a dose of about 100 mg/kg body weight, about 200 mg/kg body weight, about 250 mg/kg body weight, or about 300 mg/kg body weight.

17. The method of claim 1, wherein said composition further comprises an excipient.

18. The method of claim 17, wherein said excipient is artificial.

19. The method of claim 1, wherein said composition is combined with a food, beverage or nutritional supplement.

20. The method of claim 1, wherein said subject is human.
